(19) United States
(12) Patent Application Publication
(10) Pub. No.: US 2009/0181092 A1
(43) Pub. Date: Jul. 16, 2009

(54) Methods for Treating Joints and Discs with a Carrier Matrix and Cells

(75) Inventors: Kevin Thorne, Austin, TX (US); Brian Burkinshaw, Pflugerville, TX (US)

Correspondence Address: ANDREWS KURTH LLP, 1350 I STREET, N.W., SUITE 1100, WASHINGTON, DC, 20005, US

(73) Assignee: Spinal Restoration, Inc.

(21) Appl. No.: 12/382,217

(22) Filed: Mar. 11, 2009

Related U.S. Application Data
(63) Continuation-in-part of application No. 11/181,677, filed on Jul. 14, 2005.
Application No. 11/181,677 is a continuation-in-part of application No. 11/205,760, filed on Aug. 17, 2005.
Application No. 11/205,760 is a continuation-in-part of application No. 11/205,775, filed on Aug. 17, 2005.
Application No. 11/205,775 is a continuation-in-part of application No. 11/205,784, filed on Aug. 17, 2005.
Application No. 11/205,784 is a continuation-in-part of application No. 11/650,306, filed on Jan. 5, 2007.
Application No. 11/650,306 is a continuation-in-part of application No. 11/205,760, filed on Aug. 17, 2005.
Application No. 11/205,760 is a continuation-in-part of application No. 11/205,784, filed on Aug. 17, 2005.
Application No. 11/205,784 is a continuation-in-part of application No. 11/205,775, filed on Aug. 17, 2005.
Application No. 11/205,775 is a continuation-in-part of application No. 11/650,398, filed on Jan. 5, 2007.
Application No. 11/650,398 is a continuation-in-part of application No. 11/205,760, filed on Aug. 17, 2005.
Application No. 11/205,760 is a continuation-in-part of application No. 11/205,784, filed on Aug. 17, 2005.
Application No. 11/205,784 is a continuation-in-part of application No. 11/205,775, filed on Aug. 17, 2005.
Application No. 11/205,775 is a continuation-in-part of application No. 11/707,769, filed on Feb. 16, 2007.
Application No. 11/707,769 is a continuation-in-part of application No. 11/205,775, filed on Aug. 17, 2005.
Application No. 11/205,775 is a continuation-in-part of application No. 11/205,784, filed on Aug. 17, 2005.
Application No. 11/205,784 is a continuation-in-part of application No. 11/650,306, filed on Jan. 5, 2007.
Application No. 11/650,306 is a continuation-in-part of application No. 11/650,398, filed on Jan. 5, 2007.
Application No. 11/650,398 is a continuation-in-part of application No. 11/802,642, filed on May 24, 2007.
Application No. 11/802,642 is a continuation-in-part of application No. 11/181,677, filed on Jul. 14, 2005.
Application No. 11/181,677 is a continuation-in-part of application No. 11/892,218, filed on Aug. 21, 2007.
(60) Provisional application No. 60/588,550, filed on Jul. 16, 2004.
(60) Provisional application No. 60/623,600, filed on Oct. 29, 2004.
(60) Provisional application No. 60/623,600, filed on Oct. 29, 2004.
(60) Provisional application No. 60/623,600, filed on Oct. 29, 2004.
(60) Provisional application No. 60/623,600, filed on Oct. 29, 2004.
(60) Provisional application No. 60/764,019, filed on Feb. 1, 2006.
(60) Provisional application No. 60/854,413, filed on Oct. 24, 2006.
(60) Provisional application No. 60/623,600, filed on Oct. 29, 2004.
(60) Provisional application No. 60/764,020, filed on Feb. 1, 2006.
(60) Provisional application No. 60/854,413, filed on Oct. 24, 2006.

(51) Int. Cl. A61K 9/10; A61K 35/12

(52) U.S. Cl. 424/484; 424/93.7

(57) ABSTRACT

Methods for enhancing pain relief and accelerating repair of painful disruptions associated with damaged and/or degenerated synovial joints and spinal discs are disclosed. These methods include injecting an effective amount of a biocompatible matrix or biocompatible polymeric compound, as well as a plurality of cells, into and/or around the damaged and/or degenerated synovial joints or spinal discs. The plurality of cells may include fibroblast cells, chondrocytes, genetically altered cells, stem cells such as mesenchymal stem cells, or combinations thereof.

### RELATED APPLICATIONS

This application is a continuation-in-part of (1) U.S. patent application Ser. No. 11/181,677, filed Jul. 14, 2005, which claims priority to U.S. Provisional Application Ser. No. 60/588,550, filed Jul. 16, 2004; (2) U.S. patent application Ser. No. 11/205,760, filed Aug. 17, 2005, which claims priority to U.S. Provisional Application No. 60/623,600, filed Oct. 29, 2004; (3) U.S. patent application Ser. No. 11/205,775, filed Aug. 17, 2005, which claims priority to U.S. Provisional Application Ser. No. 60/623,600, filed Oct. 29, 2004; (4) U.S. patent application Ser. No. 11/205,784, filed Aug. 17, 2005, which claims priority to U.S. Provisional Application Ser. No. 60/623,600, filed Oct. 29, 2004; (5) U.S. patent application Ser. No. 11/650,306 filed Jan. 5, 2007, which is a continuation-in-part of U.S. patent application Ser. No. 11/205,760, filed Aug. 17, 2005, of U.S. patent application Ser. No. 11/205,784, filed Aug. 17, 2005; and of U.S. patent application Ser. No. 11/205,775, filed Aug. 17, 2005, and which claims priority to U.S. Provisional Application Ser. No. 60/623,600, filed Oct. 29, 2004, to U.S. Provisional Application Ser. No. 60/764,019, filed Feb. 1, 2006; and to U.S. Provisional Application Ser. No. 60/854,413, filed Oct. 24, 2006; (6) U.S. patent application Ser. No. 11/650,398, filed Jan. 5, 2007, which is a continuation-in-part of U.S. application Ser. No. 11/205,760, filed Aug. 17, 2005, of U.S. application Ser. No. 11/205,784, filed Aug. 17, 2005, and of U.S. application Ser. No. 11/205,775, filed Aug. 17, 2005, and which claims priority to U.S. Provisional Application Ser. No. 60/623,600, filed Oct. 29, 2004; to U.S. Provisional Application Ser. No. 60/764,020, filed Feb. 1, 2006; and to U.S. Provisional Application Ser. No. 60/854,413, filed Oct. 24, 2006; (7) U.S. patent application Ser. No. 11/707,769, filed Feb. 16, 2007; which is a continuation-in-part of U.S. patent application Ser. No. 11/205,775, filed Aug. 17, 2005; of U.S. patent application Ser. No. 11/205,784, filed Aug. 17, 2005; of U.S. patent application Ser. No. 11/650,306, filed Jan. 5, 2007; and of U.S. patent application Ser. No. 11/650,398, filed Jan. 5, 2007; (8) U.S. patent application Ser. No. 11/802,642, filed May 24, 2007, which is a continuation-in-part of U.S. patent application Ser. No. 11/181,677, filed Jul. 14, 2005; and (9) U.S. patent application Ser. No. 11/892,218, filed Aug. 21, 2007, all of which are incorporated herein by reference.

### TECHNICAL FIELD

The technical field relates to medical treatments and, in particular, to treatments for damaged or degenerated soft tissues and joints of the musculoskeletal system.

### BACKGROUND

Degenerated and damaged soft tissues of the musculoskeletal system cause and increase the risk of medical complications resulting in intense pain and restricted motion. For example, degenerated and damaged soft tissues of the spine represent the major source of back pain for millions of people around the world. Soft tissue degeneration of the ligaments and intervertebral discs also increase the risk of damage to and back pain from local spinal joints, including: zygapophysical (facet), costovertebral, sacroiliac, sacral vertebral and atlantoaxial joints.

At present, conservative treatments for damaged and/or degenerated soft tissues and joints include anti-inflammatory medications, muscle relaxants, physical therapy, and direct injections into the joints. When anti-inflammatory medications, muscle relaxants and physical therapy fail to provide pain relief, injection of the painful joint with a local anesthetic and/or steroids may also be necessary. If there is temporary relief and no surgically correctable problem, the nerves which supply sensation to the joint can be disrupted by radiofrequency ablation. Often, significant damage or degeneration necessitates more invasive, surgical therapies to repair, augment and/or replace the affected tissues and/or joint(s). For example, a common surgical solution for chronic discogenic back pain includes the removal of the disc followed by intervertebral body fusion of the motion segment. Because of the risks of surgical complications, moderate long-term pain relief benefits and accelerated adjacent tissue degeneration, there exists a significant need for more effective and less intrusive therapeutic procedures that provide pain relief caused by damaged soft tissues in degenerated joints.

### SUMMARY

Methods for enhancing pain relief and accelerating repair of painful disruptions associated with damaged and/or degenerated synovial joints and spinal discs are disclosed. Embodiments include injecting an effective amount of a biocompatible matrix or biocompatible polymeric compound, as well as a plurality of cells, into and/or around the damaged and/or degenerated synovial joints or spinal discs. The plurality of cells may include fibroblast cells, chondrocytes, genetically altered cells, stem cells such as mesenchymal stem cells, or combinations thereof.

Also disclosed are methods describing the control variables for optimizing the delivery and reaction of the product precursors, initiators and catalysts.

### DESCRIPTION OF THE DRAWINGS

The detailed description will refer to the following drawings, wherein like numerals refer to like elements, and wherein:

FIG. 1 illustrates an embodiment of a delivery device for injecting fluids into a spinal disc or synovial joint.

FIG. 2 is a flow chart showing a method for injecting fibrin into a spinal disc.

FIGS. 3A and 3B are fluoroscopy x-rays (discography) of a spinal disc before and after treatment.

FIG. 4 is a fluoroscopic x-ray of a zygapophysical joint injection.

### DETAILED DESCRIPTION

The healing of soft tissues results from a progression of events initiated by injury and directed toward reestablishing tissue structure and function. Soft tissue repair is ordinarily described as taking place in three distinct and overlapping stages: an inflammatory phase, a granulation tissue (proliferative) phase and a matrix remodeling phase. The ubiquity of proteoglycans in mammalian tissues virtually guarantees their involvement in tissue restitution through wound healing.

Normally, extravasation of blood into the wound site leads to clot formation and the development of a temporary fibrin matrix. The fibrin matrix provides temporary scaffolding that permits the ingrowth of new cells. After several days, (3-4 in vascularized tissues), fibroblasts and neovascular endothelium, in conjunction with the structural and chemotactic secretory products released by these cells, constitute a distinct entity known as granulation tissue. Granulation tissue is a fibrovascular connective tissue whose functional life begins with the establishment of the fibrin clot and ends with the formation of a healed scar. The essential transformation from granulation tissue into scar involves matrix remodeling—a process in which proteoglycans play a significant role. Remodeling continues until healing tissue produces the dense collagen architecture of the fibrotic scar. This transformation is accompanied by the production and breakdown of large quantities of glycosaminoglycan, proteoglycan, fibronectin and collagen.

The inflammatory phase of tissue repair is a leukocyte driven response to injury directed at eliminating pathogens and damaged tissue. It begins at the time of injury. A key response of endothelium to injury is cellular retraction and loss of attachments with adjacent cells. The net effect is to induce circulating platelets to adhere to newly exposed surfaces, to aggregate and to form a hemostatic plug. The activated platelets undergo both structural and functional changes leading to the release of chemotactic and mitogenic factors. These factors promote platelet aggregation and mediate the transition of fibrinogen into fibrin. The deposition of fibrin generates a dense fibrous matrix capable of entrapping cells and binding extracellular components. The fibrin clot seals the injury site, prevents additional bleeding and directs cellular proliferation, migration and repair of the tissue damage.

Proteoglycans play a fundamental role in tissue repair during the early stage of healing. Fibrin preferentially binds hyaluronan, generating a scaffold hospitable to peripheral neutrophils, monocytes, macrophages and fibroblasts. The activity and migration of these cells promote endothelial neovasculaization, innervation and granulation tissue production.

Methods for providing pain relief and enhancing the healing of painful disruptions associated with damaged or degenerated joints and/or soft tissues are disclosed. The methods describe percutaneously injecting into, around and/or on the damaged joint and/or soft tissues an effective amount of an in situ curable tissue matrix comprised of a biocompatible matrix, a biocompatible polymeric compound or components and combinations thereof. The damaged or degenerated joint can include those described anatomically as cartilaginous and/or synovial type joints. Examples of cartilaginous joints include, but are not limited to, spinal discs, the pubic symphysis, manubriostemal joints and first manubriocostal joints. Examples of synovial joints include, but are not limited to, zygapophysical joints, costovertebral joints, sacroiliac joints, sacral joint, atlantoaxial joints, hand joints (e.g., thumb), wrist joints (e.g., carpals); elbow joints, shoulder joints, temporomandibular (TMJ) joints, sacroiliac joints, hip joints, knee joints, ankle, and foot joints. The damaged or degenerated soft tissues can include muscles, tendons ligaments, cartilage, meniscal and labrum tissue.

### Biocompatible Matrix and Biocompatible Polymeric Compound

As used hereinafter, the term “biocompatible matrix” refers to material scaffolds of interconnected open porosity that are cytocompatible and stimulate minimal inflammation or immune responses when incorporated into a living being (e.g., humans). The methods describe the formation and delivery of tissue healing scaffolds to the damaged or degenerated joint or soft tissue. Biological remodeling of the matrix scaffold depends, in part, upon the ability of cells to migrate into the matrix from the surrounding tissues and produce repair and or regeneration of the tissue defect. Accordingly, the structural and biochemical characteristics of the matrix may be further optimized to promote specific chemical, nutritional or tissue migration. Although the mechanical and biological performance of some tissue matrix scaffolds are well known to those familiar with the art, achieving the ultimately desired combination of properties represents a technological challenge that has yet to be achieved.

As used hereinafter, the term “biocompatible polymeric compound” refers to porous and nonporous polymeric compounds that are cytocompatible, biologically inert, non-inflammatory, nontoxic and generate minimal immune reaction when incorporated into a living being (e.g., humans).

The biocompatible matrix or biocompatible polymeric compound can be non-degradable or degradable. A “degradable polymeric compound” is a polymeric compound that can be degraded and absorbed in situ in a living being such as human.

In preferred embodiments, the biocompatible matrix or biocompatible polymeric compound will either permanently or temporarily augment the damaged and degenerated tissues to restore functionality. The material should also function as a porous scaffold possessing physicochemical properties suitable for use in the repair and regeneration of musculoskeletal soft tissues (tendons, cartilage and fibrotic scar tissue). The scaffold material can be naturally derived or synthetic and should be formed in situ in the presence of cells and tissues. The scaffolds must also satisfy the requirements for cellular tissue repair. This requires precise control of porosity and internal pore architecture to ensure blood flow and adequate diffusion of nutrients and interstitial fluid, optimize cell migration, growth and differentiation and maximize the mechanical function of the scaffolds and the regenerated tissues.

Examples of naturally derived compositions include, but are not limited to, fibrin, collagen (e.g., Type I, II, and III collagen), fibronectin, laminin, polysaccharides (e.g., chitosan), polycarbohydrates (e.g., porteoglycans and glycosaminoglycans), cellulose compounds (e.g., methyl cellulose, carboxymethyl cellulose, and hydroxy-propylmethyl cellulose) and combinations thereof. Examples of synthetic compositions that satisfy these requirements include, but are not limited to, aliphatic polyesters (e.g., polylactides (PLA), polycaprolactone (PCL) and polyglycolic acid (PGA)), polyglycols (e.g., polyethylene glycol (PEG), polymethylene glycol, polytrimethylene. glycols), polyvinyl-pyrrolidones, polyanhydrides, polyethylene oxide (PEO), polyvinyl alcohols (PVA), poly(thyloxazoline) (PEOX), polyoxyethylene and combinations and derivatives thereof. The biocompatible matrix and biocompatible polymeric compound may be obtained autologously or supplemented endogenously with host body fluids to increase their biocompatibility with host tissues.

### Fibrin Embodiments

In a preferred embodiment, the in situ curable, degradable biocompatible matrix is fibrin. The formation of fibrin mimics the final stage of the natural clotting mechanism. Fibrin formation is initiated following activation of fibrinogen by a fibronogen activating agent such as thrombin and reduction of fibrinogen into fibrinopepetides. The fibrinopeptides spontaneously react and polymerize into fibrin. Fibrinogen can be isolated from autologous (i.e., from the patient to be treated), heterologous (i.e., from other human, pooled human supply, or non-human sources) tissues or recombinant sources. Fibrinogen can be provided in fresh or frozen solutions. Fibrinogen is also commercially provided in a freeze-dried form. Freeze-dried fibrinogen is typically reconstituted in a solution containing aprotinin (a polyvalent protease inhibitor which prevents premature degradation of the formed fibrin). Aprotinin may be derived from autologous and heterologous tissues, recombinant sources and synthetic chemical laboratories. Freeze-dried fibrinogen, thrombin and aprotinin are available in kit form from manufacturers such as Baxter under names such as TISSEEL®.

Fibrinogen is biomedically used in a concentration range of 50-150 mg/ml. In a preferred embodiment, freeze-dried fibrinogen is reconstituted at a concentration between 75-115 mg/ml. A polyvalent protease inhibitor-free reconstituting solution is preferrably used to reconstitute fibrinogen. For effective protease inhibition, aprotinin is used in concentrations ranging between 2000-4000 KIU/ml. In the preferred embodiment, the reconstitution solution contains aprotinin at a concentration of 3000 KIU/ml.

The amount of fibrinogen activating agent can be varied to alter its macrostructure and to reduce or lengthen the time to complete fibrin formation. Examples of fibrinogen activating agents include, but are not limited to, thrombin and thrombin-like enzymes. Thrombin is an enzyme that converts fibrinogen to fibrin. Thrombin can be isolated from autologous, heterologous tissues or recombinant sources. Thrombin can be provided in fresh or frozen solutions. Thrombin is also commercially available in freeze-dried form.

Thrombin is typically used in the range 30-70 mg/ml to rapidly solidify fibrin into a interconnected porous scaffold. In a preferred embodiment, the freeze-dried thrombin is reconstituted to a final concentration of about 45-55 mg/ml. The reconstitution solution preferably contains calcium chloride in the range of about 1 to 100 mmol/ml as required to activate thrombin and initiate fibrin formation.

Thrombin-like enzymes also initiate the release of fibrinopeptides from fibrinogen and stimulate the formation of fibrin. Thrombin-like enzymes are commonly isolated from the venom of several poisonous snakes and poisonous marine life (e.g., jellyfish, sea snakes, cone shells, and sea urchins). Depending on its composition and source, the thrombin-like enzyme may preferentially reduce fibrinogen with the release of fibrinopeptide A and B at different rates. TABLE 1 is a non-limiting list of the sources of the snake venoms that can be used with the herein disclosed methods, the name of the thrombin-like enzyme, and which fibrinopeptide(s) is released by treatment with the enzyme.

TABLE 1
Commonly used snake venoms

| | | Fibrinopeptide |
|---|---|---|
| Source | Name | Released |
| Agkistrodon acutus | Acutin | A |
| A. contortrix contortrix | Venzyme | B, (A)* |
| A. halys pallas | | B, (A)* |
| A. (Calloselasma) | Ancrod, Arvin | A |
| rhodostoma | | |
| Bothrops asper | Asperase | A |
| B. atrox, B. moojeni, | Batroxobin | A |
| B. maranhao | | |
| B. insularis | Reptilase | A, B |
| B. jararaca | Botropase/bothrombin | A |
| Lachesis muta muta | Defibrase | A, B |
| Crotalus adamanteus | Crotalase | A |
| C. durissus terrificus | | A |
| Trimeresurus flavoviridis | Flavoxobin/habutobin | A |
| T. gramineus | Grambin | A |
| Bitis gabonica | Gabonase | A, B |

( )* means low activity.

For a review of thrombin-like enzymes from snake venoms, see H. Pirkle and K. Stocker, Thrombosis and Haemostasis, 65(4):444-450 (1991), which is incorporated herein by reference. The preferred thrombin-like enzymes are Batroxobin, especially from B. moojeni, B. maranhao and B. atrox; and Ancrod, especially from A. rhodostoma.

In general, higher concentrations of thrombin or thrombin-like enzyme per unit amount of fibrinogen stimulate faster fibrin formation. The relative concentrations of fibrinogen, thrombin and/or thrombin-like enzyme and calcium are important for controlling the viscosity of the combined components, the ease of mixing and delivery, the rate of fibrin formation and the mechanical properties of the fibrin product. In addition, the aggressiveness of component mixing plays a significant role in fibrin's setting duration. The method of mixing and delivery can also have a significant effect on the micro-porous structure, biological degradation resistance and mechanical function of the fibrin product. Proper control of these variables is required to ensure that fibrin has time to flow into the complex biologic tissue anatomy prior to setting and that the product possesses the structural, mechanical and physiological properties necessary for tissue repair.

Delivery for any of the described biocompatible matrices, biocompatible polymeric compounds or additives can be achieved by percutaneous injection into the tissue or joint under direct visualization or with fluoroscopic control, or by direct injection into the tissue or joint in an open, mini-open or endoscopic procedure.

### Biological Additives

The biocompatible matrix or biocompatible polymeric compound may be administered or combined with one or more additives to reduce pain and/or enhance joint and tissue healing. As used herein, the term “biological additives” includes: anesthetics and/or analgesics (e.g., lidocaine and bupivicaine); proteoglycans (e.g., sGAG, aggrecan, chondrotin sulfate, deratin sulfate, versican, decorin, fibronectin and biglycan); hyaluronic acid and salts and derivatives thereof; pH modifiers and buffering agents; anti-oxidants (e.g., superoxide dismutase, and melatonin); protease inhibitors (e.g., TIMPtypes I, II, III); cell differentiation and growth factors that promote healing and tissue regeneration (e.g., TGF-β, PDGF, BMP-2,6,7, LMP-1, and CSF); biologically active amino acids, peptides, and derivatives thereof (e.g., fibroblast attachment peptides such as Arg-Gly-Asp, (RGD), Arg-Gly-Asp-Ser (RGDS), Gly-Arg-Gly-Asp-Ser (GRGDS), P-15 and fibroblast migration peptides such as Met-Ser-Phe (MSF) and Ile-Gly-Asp (IGD), and Gly-Asx-Asp (GBD)); anti-inflammatory agents (e.g., erythropoietin-corticosteroid); antibiotics; antifungals; antiparasitics; histamines; antihistamines; anticoagulants; vasoconstrictors, vasodilators; vitamins; cellular nutrients (e.g., glucose and other sugars); gene therapy reagents (e.g., viral and non-viral vectors); salicylic acid and derivatives of salicylic acid (e.g., acetylsalicylic acid).

Any of the aforementioned additives may be added to the biocompatible matrix or biocompatible polymeric compound separately or in combination. For example, one or more of these additives can be injected with the biocompatible matrix or biocompatible polymeric compound. Combinations of these additives can be employed and different additives can be used in the solutions that are used to reconstitute the biocompatible matrix or biocompatible polymeric compound. For example, a solution containing a local anesthetic and/or glucosamine sulfate may be used to reconstitute the fibrinogen, and a solution containing type II collagen may be used to reconstitute the activating agent. Likewise, one or more of these additives can be injected separately, either before or after the injection of the fibrin. For solutions containing an incompletely water-soluble additive, an anti-caking agent such as polysorbate, may be added to facilitate suspension of this additive.

In one embodiment, the additive is a buffering agent that maintains the pH of the fibrin solution within the physiological range of pH 7-8.

In one embodiment, the additive is an analgesic or anesthetic. The amount and type of anesthetic used should be chosen so as to be effective in alleviating the pain of injection when the biocompatible matrix or biocompatible polymeric compound is injected or otherwise introduced into the joint or surrounding structures. Representative analgesics and anesthetics include, but are not limited to, lidocaine (alpha-diethylaminoaceto-2,6-xylidide), SARAPIN (soluble salts and bases from Sarraceniaceae (Pitcher Plant)), bupivacaine (1-butyl-N-(2,6-dimethylphenyl)-2-piperidinecarboxamide) and procaine (2-diethylamino ethyl 4-aminobenzoate hydrochloride). Combinations of analgesics and/or anesthetics also can be used. Anesthetics may be long-acting or short-acting in their duration and effect.

In another embodiment, the additive is a growth-inductive protein that enhances tissue growth and promotes rehabilitation of the damaged tissues.

In another embodiment, the additive is a nutrient that enhances cell growth.

In yet another embodiment, the additive is salicylic acid or a derivative of salicylic acid.

### Cellular Additives

The biocompatible matrix or biocompatible polymeric compound may also be administered with one or more cellular and biological additives to enhance joint and tissue healing.

As used herein, the term “cellular additives” includes any kind of cells that could assist in the repair of the damaged or degenerated joint and/or tissue. Appropriate cells include, but are not limited to, autologous fibroblasts from dermal tissue, oral tissue, or mucosal tissue; autologous chondrocytes or fibroblasts from tendons, ligaments or articular cartilage sources; allogenic juvenile or embryonic chondrocytes; stem cells such as mesenchymal stem cells and embryonic stem cells; and genetically altered cells. Stem cells can be autologous or allogenic. Precursor cells of chondrocytes, differentiated from stem cells, can also be used in place of the chondrocytes. As described herein, the term “chondrocytes” includes chondrocyte precursor cells.

In one embodiment, fibrin or other in situ curable, biocompatible matrix or biocompatible polymeric compound is premixed with a cellular additive prior to injection. In another embodiment, the fibrin or other in situ curable, biocompatible matrix or biocompatible polymeric compound is mixed with a cellular additive during the injection. In another embodiment, the fibrin or other in situ curable, biocompatible matrix or biocompatible polymeric compound is injected first, followed with an injection of a cellular additive. In yet another embodiment, a cellular additive is injected first, followed with an injection of fibrin or other in situ curable, biocompatible matrix or biocompatible polymeric compound. In all cases, fibrin or other in situ curable, biocompatible matrix or biocompatible polymeric compound functions as a matrix scaffold for cell proliferation, migration and matrix formation at or around the injection site. The injection of cells is performed under physiologic conditions to maintain cell viability.

The injected cells may be harvested, morselized and prepared pre-operatively or intra-operatively through various techniques known in the art. Fibroblasts can be obtained from a biopsy specimen. In one embodiment, a biopsy specimen is washed repeatedly with antibiotic and antifungal agents. The epidermis and the subcutaneous adipocyte-containing tissue is removed, so that the resultant culture is substantially free of non-fibroblast cells. The dermis is divided into fine pieces with scalpel or scissors. The pieces of the specimen are individually placed with a forceps onto the dry surface of a tissue culture flask and allowed to attach for between 5 and 10 minutes before a small amount of culture medium is slowly added, taking care not to displace the attached tissue fragments. After 24 hours of incubation, the flask is fed with additional medium. The establishment of a cell line from the biopsy specimen ordinarily takes between 2 and 3 weeks, at which time the cells can be removed from the initial culture vessel for expansion.

During the early stages of the culture, it is desired that the tissue fragments remain attached to the culture vessel bottom. Fragments that detach should be reimplanted into new vessels. In one embodiment, the fibroblasts can be stimulated to grow by a brief exposure of the tissue culture to EDTA-trypsin, according to techniques well known to those skilled in the art. The exposure to trypsin is too brief to release the fibroblasts from their attachment to the culture vessel wall. Immediately after the cultures have become established and are approaching confluence, samples of the fibroblasts can be removed for frozen storage. The frozen storage of early rather than late passage fibroblasts is preferred because the number of passages in cell culture of normal human fibroblasts is limited prior to cellular dedifferentiation.

The fibroblasts can be frozen in any freezing medium suitable for preserving fibroblasts. In one embodiment, the freezing medium consists of 70% growth medium, 20% (v/v) fetal bovine serum and 10% (v/v) dimethylsulfoxide (DMSO). Thawed cells can be used to initiate secondary cultures to obtain suspensions for use in the same subject without the inconvenience of obtaining a second specimen.

Any tissue culture technique that is suitable for the propagation of dermal fibroblasts from biopsy specimens may be used to expand the cells to practice the invention. Techniques well known to those skilled in the art can be found in R. I. Freshney, Ed., ANIMAL CELL CULTURE: A PRACTICAL APPROACH (IRL Press, Oxford England, 1986) and R. I. Freshney, Ed., CULTURE OF ANIMAL CELLS: A MANUAL OF BASIC TECHNIQUES, Alan R. Liss & Co., New York, 1987), which are hereby incorporated by reference.

Similarly, chondrocytes can be obtained from another site in the patient or from autopsy, using for example, cartilage obtained from joints or rib regions. The cartilage is sterilized, for example, by washing in Povidone-lodine 10% solution (Betadine, Purdue Frederick Co., Norwalk, Conn.). Then, under sterile conditions, the muscle attachments are dissected from the underlying bone to expose the joint surfaces. The cartilage from the articulating surfaces of the joint is sharply dissected from the underlying bone, cut into pieces with dimensions of less than 5 mm per side, and washed in Phosphate Buffered Saline (PBS) with electrolytes and adjusted to neutral pH. The minced cartilage is then incubated at 37° C. in a collagenase solution and agitated overnight (e.g., as described by Klagsbrun, Methods in Enzymology, Vol. VIII). This suspension is then filtered using a nylon sieve (Tetko, Elmford, N.Y. 10523). The cells are then removed from the suspension using centrifugation, washed twice with PBS solution and counted with a hemocytometer. The solution is centrifuged at 1800 rpm and the supernatant above the cell suspension removed via suction using a micropipette until the volume of the solution yields a chondrocyte concentration of 5×10⁷ cells/ml.

The isolated chondrocytes can be cultured in a suitable culture medium at 37° C. In one embodiment, the culture medium is Hamm's F-12 culture medium with 10% fetal calf serum, L-glutamine (292 μg/ml), penicillin (100 U/ml), streptomycin (100 μg/ml) and ascorbic acid (5 μg/ml).

In another embodiment, the cells are mesenchymal stem cells. Mesenchymal stem cells are multipotent stem cells that can differentiate into a variety of cell types, including osteoblasts, chondrocytes, myocytes, and neuronal cells. Mesenchymal stem cells may be isolated from fat, bone marrow, umbilical cord blood, or placenta. Methods for isolating mesenchymal stem cells from each of these sources are well known to one skilled in the art.

In another embodiment, the cells are pluripotent stem cells from adult human testis. Such cells may be isolated as described by Conrad et al. (Conrad et al., “Generation of pluripotent stem cells from adult human testis” Nature. 2008, 456:344-349, which is hereby incorporated by reference).

### Injection Device

The biocompatible matrix or biocompatible polymeric compound may be injected as monomers, activated monomers or low molecular weight reactive polymers that are activated, polymerized and/or cross-linked at the injection site (in situ curable). In essence, the injected, in situ curable, biocompatible matrix or biocompatible polymeric compound would quickly set into an elastic coagulum and provide a conductive tissue scaffold with a biologic milieu that may help tissue repair, joint hydration and joint health restoration. In the case of spinal disc injection, the injected, in situ curable, biocompatible matrix or biocompatible polymeric compound would also provide (at least temporarily) limited restoration of joint height.

The term “injecting” as used herein therefore encompasses any injection of a biocompatible matrix, a biocompatible polymeric compound, or components that form the biocompatible matrix, or the biocompatible polymeric compound in a joint/tissue or surrounding structures, including circumstances where a portion of the components are mixed and reacted to initiate biocompatible matrix or biocompatible polymeric compound formation prior to contact with or actual introduction into the joint or tissue. The herein disclosed methods also describe the sequential injection of the reactive components for formation of a biocompatible matrix or biocompatible polymeric compound into the joint, tissue or surrounding structures. For example, thrombin or thrombin-like enzymes can be injected followed by the injection of fibrinogen. The components can also be injected in reverse order or intermittently injected into the joint/tissue or surrounding structures. Additional additives may be incorporated into the components and further mixed into the fibrin during injection. The term “injecting” as used herein also encompasses percutaneous injection into the tissue or joint, under direct visualization or with fluoroscopic control, and direct injection into the tissue or joint in an open, mini-open or endoscopic procedure.

In one embodiment, a dual-syringe injector is used and the mixing of the components that form the biocompatible matrix or the biocompatible polymeric compound at least partially occurs in the Y-connector and in the needle mounted on the Y-connector, with the balance of the clotting occurring in the joint/tissue or surrounding structures. This method of preparation facilitates the formation of the biocompatible matrix or the biocompatible polymeric compound at the desired site in the joint/tissue or surrounding structures during delivery, or immediately thereafter. Examples of dual syringe injection devices are described in U.S. Pat. No. 4,874,368 and U.S. Patent Application Publication No. 20070191781, which are hereby incorporated by reference in their entirety. A person of ordinary technical expertise would understand that other injecting devices may be used to efficiently mix different components during injection. For example, the Y-connector may be replaced with a coaxial needle. Multi-syringe injectors having more than two syringes may also be used.

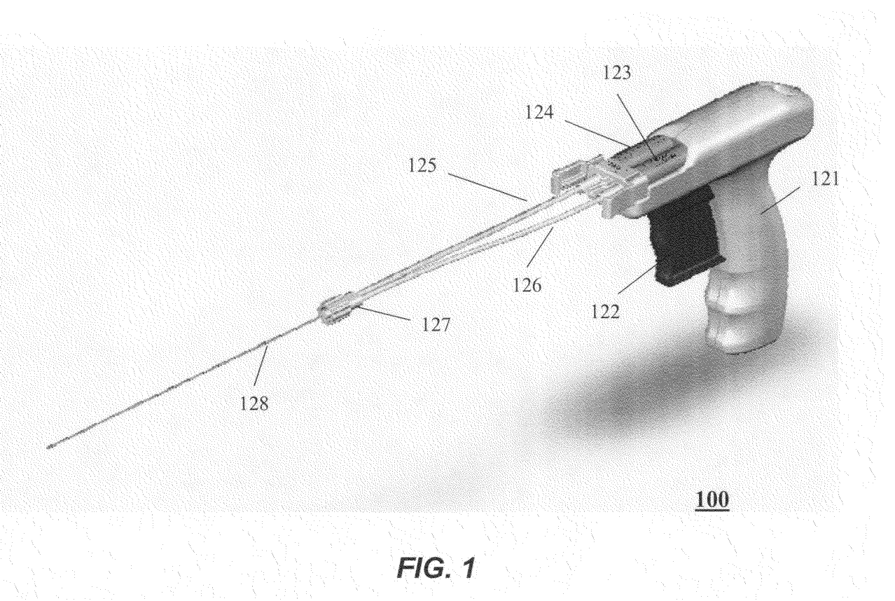

In one embodiment, fibrin is injected using a delivery device such as that shown in FIG. 1. In this embodiment, the delivery device 100 includes main housing 121 into which are inserted fibrinogen capsule 123 and thrombin capsule 124. Trigger 122, in conjunction with a pressure monitor (not shown) controls injection of the fluids. Attached to the capsules 123, 124 is an inner needle assembly including delivery tubes 125 and 126, (connected to an inner, coaxial needle, (not shown), within the outer needle 128). Connector 127 serves to connect the delivery tubes 125, 126 and the inner coaxial needle to the outer needle 128. One skilled in the art would understand that the above-described injection procedures and delivery devices, including the delivery device 100, also apply to injection of other biocompatible matrix or biocompatible polymeric compounds.

### Injection Procedure

Depending on the location of the joint, the biocompatible or polymeric matrix may be delivered during open surgical exposures or by percutaneous injection. Percutaneous injections may be performed under fluoroscopic visualization or under direct visualization. Injection of the biocompatible or polymeric matrix into (within) blood vessels is to be avoided.

Preferably, a non-iodinated contrast agent may be used in conjunction with the injection of the biocompatible matrix or the biocompatible polymeric compound to ensure the correct placement at the site and avoidance of blood vessels. The contrast agent may be injected prior to injection of the biocompatible matrix or the biocompatible polymeric compound. In the case of fibrin, the contrast agent may be included in the fibrinogen component or the activating agent component that is injected into the joint or tissue. Contrast agents and their use are well known to those skilled in the art.

In a preferred embodiment, the injection point is in the nucleus pulposus or within the annulus fibrosus of a spinal disc. If the injection occurs in the nucleus pulposus, the injected components may form a patch at the interface between the nucleus pulposus and the annulus fibrosus, or, more commonly, the components flow into the defect(s) (e.g., fissures) of the annulus fibrosus and potentially “overflow” into the extradiscal space. Over-pressurizing the disc beyond natural physiologic pressure ranges when injecting the components into the disc, should be avoided to limit extradiscal leakage and reduce annulus fibrosus damage.

If the injection occurs in a zygapophysical joint, the injected components may form a patch at the interface between the facets, and/or within the fibrous tissues of the joint between the superior articular process of one (lower) vertebra and the inferior articular process of the adjacent (higher) vertebra.

Because many surrounding tissues are often damaged during surgery, other embodiments encompass the delivery of the biocompatible polymeric matrix to the tissues surrounding the synovial joint, including neighboring muscles, tendons and/or ligaments. The biocompatible matrix or biocompatible polymeric compound can also be injected to reduce negative consequences and enhance the healing of surgical damage. The biocompatible matrix or biocompatible polymeric compound can also be injected around a damaged or degenerated joint to cover or coat exposed nerve ends, therefore reducing pain associated with the damaged or degenerated joint.

In preferred embodiments, the injection of fibrin or other biocompatible matrix or biocompatible polymeric compound is performed immediately before or after a surgical procedure designed to treat the damaged or degenerated joint. The injection time is determined by the attending physician based on the nature and extent of the surgical procedure, the in vivo mixing and curing/setting times, the condition of the joint, and other patient concerns.

In other embodiments, a joint that is at high risk of being damaged or of degeneration, such as spinal disc or a zygapophysical joint located next to a damaged or degenerated spinal disc, is prophylactically treated to delay or prevent the development of permanent or irreversible degenerative changes in the joint. The effect of the treatment, such as re-hydration of a dehydrated joint, may be monitored using T2-weighted magnetic resonance imaging (MRI). In the presence of any ferro-magnetic implants, a CT or x-ray image could be utilized to evaluate changes in bone anatomy.

In other embodiments, the injection of fibrin or other biocompatible matrix or biocompatible polymeric compound is performed to augment joints and tissues following surgical repair. The joints may be repaired using any known surgical procedures. Common examples of spinal surgical procedures include, but are not limited to, conventional open discectomy, mini-open discectomy, percutaneous discectomy, laminectomy, spinal fusion, artificial disc replacements (ADR), vertebral body replacements (VBR), partial vertebral body replacements (PVBR) and combinations thereof.

In other embodiments, repaired tissues such as ligaments, tendons, torn muscles, cartilage flaps and plugs, and meniscal and labrum tissues may be augmented and secured by the direct visual or percutaneous injection of fibrin or other biocompatible matrix or biocompatible polymeric compound.

### Injection Volume

The biocompatible matrix or the biocompatible polymeric compound will generally be used in an amount effective to achieve the intended result, i.e., delay or prevent degeneration, augment tissue strength and/or repair or prevent damage of a joint and its surrounding areas. The term “effective amount” refers to a dosage sufficient to provide for treatment for the disease state being treated, to ameliorate a symptom of the disease being treated, or to otherwise provide a desired effect. The effective amount of the biocompatible matrix or the biocompatible polymeric compound administered will depend upon a variety of factors, including, for example, the type, site and size of a joint or tissue, the mode of administration, the age and weight of the patient, the bioavailability of the particular additive, and whether the desired benefit is prophylactic or therapeutic. In cases where the injection is performed concurrently with a surgical procedure to reinforce the surgically treated joint or to prophylactically reinforce structures near the treated joint, the effective amount of the biocompatible matrix or the biocompatible polymeric compound administered will also depend upon the nature of the surgical procedure. Determination of an effective dosage is well within the capabilities of those skilled in the art.

For intrajoint or intradiscal injections, the total volume of the injection may be anatomically limited. In confined joints, an injection volume of 0.20-5.00 ml of biocompatible matrix or biocompatible polymeric compound will fill most intradiscal, facet, temporomandibular (TMJ), shoulder, knee and hip joints. In damaged, leaking joints, larger injection volumes of biocompatible matrix or biocompatible polymeric compound may be required to adequately fill the desired joint. It is estimated that the injection volumes to treat external joint tissues can range from as little as 1 ml to as much as 10 ml or more.

The dosage and volume of the biocompatible matrix or the biocompatible polymeric compound, such as fibrin, may be adjusted individually to provide local concentrations of the agents that are sufficient to maintain a protective or therapeutic effect. For example, the biocompatible matrix or the biocompatible polymeric compound may be administered in a single injection or by sequential injections. The injection may be repeated periodically. Skilled artisans will be able to optimize effective local dosages and the injection regimen without undue experimentation. The dose ratio between toxic and protective/therapeutic effect is the therapeutic index. Agents that exhibit high protective/therapeutic indices are preferred.

### Injection Locations

The point, or points, of injection (e.g., at the tip of injection needle) can be both in and surrounding the joint, tissue or supporting structure. In a preferred embodiment, the biocompatible matrix or polymeric compound is injected into a damaged or degenerated spinal disc joint. Degenerative disc disease is one of today's most common and costly medical conditions. Marked by the gradual erosion of cartilage and disc degeneration between the vertebrae, this destructive spinal disease routinely provokes discogenic pain, especially in the lower back. Disc degeneration commonly occurs during aging. As people age, the nucleus pulposus begins to degenerate and lose water content, making the disc less effective as a compressive cushion and in its ability to transmit physical loads to the annulus fibrosus. As a disc continues to degenerate, the annulus fibrosus also degrades resulting in defects that can eventually grow into macroscopic tears. These defects, also known as internal disc disruptions (IDD), are known to allow the displacement of the components in the nucleus pulposus through the annulus fibrosus to the highly innervated outer ⅓ of the annulus and into the spaces occupied by the nerve roots and spinal cord (this is sometimes also called “Leaky Disc Syndrome”). IDD can act as stress concentration sites that severely weaken the structural integrity of the annulus. It is not uncommon for the tears to result, producing a herniated disc.

Another appropriate spinal disc for treatment includes the “herniated disc”. A spinal disc, having lost water content and structural integrity due to aging, or having been subjected to excessive stresses due to injury, will develop a weakened annulus fibrosus. The areas of the annulus fibrosus subjected to the highest stresses (usually near the posterior aspect of the disc) are most prone to stress injuries manifesting in the forms of tears, or herniation of the annular fiber structures. The herniation can then press on the nerves, spinal cord, and spinal nerve roots found outside the disc and cause pain, numbness, tingling and/or weakness in the extremities. Prolonged herniation may also lead to an inflammatory condition known as a chemical radiculitis.

Fibrin or other in situ curable, biocompatible matrix or biocompatible polymeric compound can be injected into the nucleus and/or anulus to reinforce and facilitate the repair of the damaged or degenerated spinal disc. In one embodiment, fibrin or other in situ curable biocompatible matrix or biocompatible polymeric compound is injected into a damaged or degenerated disc to seal and augment the repair of fissures, cracks, and voids in the anulus fibrosus. In another embodiment, fibrin or other in situ curable biocompatible matrix or biocompatible polymeric compound is used to seal, coat or fill, fissures, cracks, voids and Schmorl's nodes in an end plate of a spinal disc. In another embodiments, fibrin or other in situ curable, biocompatible matrix or biocompatible polymeric compound is injected into a damaged or degenerated spinal disc in a sufficient amount to increase disc height and relieving pressure on nerve roots near the spinal disc. In another embodiment, fibrin or other in situ curable biocompatible matrix or biocompatible polymeric compound is injected into areas surrounding a damaged or degenerated spinal disc to cover or coat exposed nerve roots around the spinal disc. In yet another embodiment, fibrin or other in situ curable biocompatible matrix or biocompatible polymeric compound is introduced into a vertebral canal or a thecal sac near a spinal disc.

In other embodiments, the damaged or degenerated joint is a zygapophysical joint. Zygapophysical joints, also called facet joints, are found at every spinal level (except at the top level) and provide about 20% of the torsional (twisting) stability in the neck and low back. Each upper half of the paired zygapophysical joints are attached on both sides on the backside of each vertebra, near its side limits, then extend downward. The other halves of the joints arise on the vertebra below, then project upwards to engage the downward faces of the upper facet halves. The zygapophysical joints slide on each other and both sliding surfaces are normally coated by a very low friction, moist cartilage. A small sack or capsule surrounds each facet joint and provides a sticky lubricant for the joint. Each sack has a rich supply of tiny nerve fibers that provide a warning when irritated.

Zygapophysical joints are in almost constant motion with the spine and commonly overloaded, worn or degenerated as the disc space narrows due to aging and disc dehydration. In these situations, the cartilage coating the facet joints may thin or disappear resulting in bone-on-bone contact and or boney facet joint abnormalities. The resulting osteoarthritis can produce considerable back pain on motion. This condition may also be referred to as “facet joint disease” or “facet joint syndrome”. Injection of fibrin or other in situ curable, biocompatible matrix or biocompatible polymeric compound into or around a damaged or degenerated zygapophysical joint may repair and/or reinforce the joint and alleviate pain associated with the damaged or degenerated zygapophysical joint.

In other embodiments, the damaged or degenerated joint is a costovertebral joint. The costovertebral joints are the articulations that connect the heads of the ribs with the bodies of the thoracic vertebrae. Joining of ribs to the vertebrae occurs at two places, the head and the tubercle of the rib. Two convex facets from the head attach to two adjacent vertebrae. Costovertebral joint has the requisite innervation for pain production in a similar manner to other joints of the spinal column and has been considered a potential source of upper back, shoulder, and atypical chest pain.

In other embodiments, the damaged or degenerated joint is a sacroiliac joint. The sacroiliac joint is the joint between the sacrum, at the base of the spine, and the ilium of the pelvis, which are joined by ligaments. It is a strong, weight bearing synovial joint with irregular elevations and depressions that produce interlocking of the bones. Damaged or degenerated sacroiliac joints often cause lower back and leg pain. Inflammation of the sacroiliac joints and associated ligaments are also very common, especially following pregnancy where natural hormones relax ligaments in preparation for childbirth.

In other embodiments, the damaged or degenerated joint is a sacral joint. The sacrum is a triangular structure at the base of the vertebral column. It is composed of five vertebrae that develop as separate structures, but gradually become fused in adulthood. The spinous processes of these bones are represented by a ridge of tubercles that form a median sacral crest. To the sides of the tubercles are rows of openings, the dorsal sacral foramina, through which an abundant supply of nerves and blood vessels pass. Below the sacrum is the coccyx, or tailbone, the lowest part of the vertebral column. It is composed of four vertebrae which typically fuse together by the age of twenty-five. However, in many individuals this fusion process in the sacrum and coccyx is disrupted when the vertebral column is subjected to forceful trauma or excessive loading, such as falling backwards into a sitting position. This may result in fracture of dislocation of these typically fused joints, sometimes resulting in partially-fused, cartilaginous or fibrotic joints. These joints can become innervated and be subject to micro-motion that subsequently irritates the innervated structures, resulting in pain and irritation.

In other embodiments, the damaged or degenerated joint is an atlanto-axial joint. The atlanto-axial joint has complicated structure comprising no fewer than four distinct joints. There is a pivot articulation between the odontoid process of the axis and the ring formed by the anterior arch and the transverse ligament of the atlas. Osteoarthritis of the atlanto-axial joint may lead to degenerative lesions and occipital head and neck pain.

In other embodiments, the treatment is injected into the tendon insertion point or the tendon repair site at the time of surgery, (e.g., Achilles tendon repair). In yet another embodiment, the treatment is injected into the muscle insertion point or the muscle repair site at the time of surgery, (e.g., rotator cuff repair). Both procedures are routinely performed in arthroscopic, mini-open and open techniques that would easily facilitate percutaneous applications of the treatment.

In still other embodiments, the treatment is injected into one of the many synovial joints previously described (e.g., hand, wrist, elbow, shoulder, TMJ, hip, knee, ankle and/or foot) to facilitate the repair or expedited regeneration of damaged tissues. As mentioned previously, the treatment may be injected into and around a cartilage, at a cartilage attachment point, beneath a cartilage flap, or into suture repair site. The treatment may also be injected into and around meniscal tissues (e.g., at a meniscus attachment point, under a flap, or into a suture repair site) or the glenoid/acetabulum labrum (e.g., at a labrum attachment point, under a flap, or into a suture repair site), to secure it to base structures and to augment the healing process.

The disclosed methods may be better understood by reference to the following examples, which are representative and should not be construed to limit the scope of the claims hereof.

### EXAMPLES

### Example 1

### Injection of Fibrin with a Dual-Syringe Injector

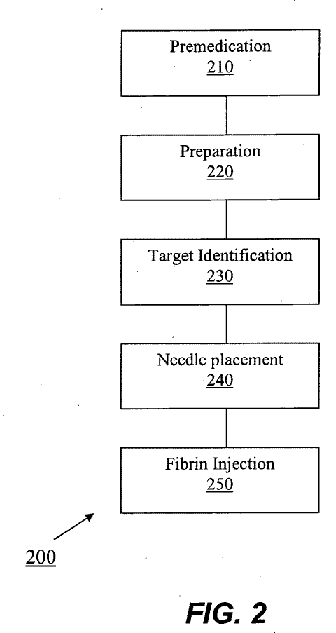

As shown in FIG. 2, injection of fibrin involves several steps, which are outlined below. The examplary method 200 is based on use of the delivery device 100 shown in FIG. 1.

### Pre-Medication (210)

As a first step, intravenous antibiotics are administered 15 to 60 minutes prior to commencing the procedure as prophylaxis against discitis. Patients with a known allergy to contrast medium should be pre-treated with H1 and H2 blockers and corticosteroids prior to the procedure in accordance with International Spine Intervention Society (ISIS) recommendations. Sedative agents may be administered but the patient should remain awake during the procedure and capable of responding to pain from pressurization of the disc. The pre-medication step may not be necessary if the fibrin sealant is injected immediately after a surgical procedure (e.g., discectomy).

### Preparation (220)

The injection procedure should be performed in a suite suitable for aseptic procedures and equipped with fluoroscopy (C-arm or two-plane image intensifier) and an x-ray compatible table to allow visualization of needle placement.

Local anesthetic for infiltration of skin and deep tissue and nonionic contrast medium with 10 mg per cc of antibiotic should be available for this procedure.

### (a) Preparation of the Fibrin Sealant

Preparation of the fibrin sealant may require approximately 25 minutes. In an embodiment, freeze-dried fibrinogen and thrombin are reconstituted in a fibrinolysis inhibitor solution and a calcium chloride solution, respectively. The reconstituted fibrinogen and thrombin solutions are then combined and mixed within the delivery device 100 to deliver and polymerize fibrin within the treated joint.

### (b) Preparation of the Delivery Device

Maintaining a sterile environment, the delivery device 100 is assembled and checked for function in preparation for the reconstituted thrombin and fibrinogen component solutions to be transferred into the device.

### (c) Patient Positioning and Skin Preparation

The patient should lie on a radiography table in either a prone or oblique position depending on the physician's preference. By means of example for a lumbar disc treatment, the skin of the lumbar and upper gluteal region should be prepared as for an aseptic procedure using non-iodine containing preparations.

### Target Identification (230)

For intradiscal injections, disc visualization and annulus fibrosus puncture should be conducted according the procedures used for provocation discography. The targeted disc should be approached from the side opposite of the patient's predominant pain. If the patient's pain is central or bilateral, the target disc can be approached from either side.

An anterior-posterior (AP) image of the lumbar spine is obtained such that the x-ray beam is parallel to the inferior vertebral endplate of the targeted disc. The beam should then be angled until the lateral aspect of the superior articular process of the target segment lies opposite the axial midline of the target disc. The path of the intradiscal needle should be parallel to the x-ray beam, within the transverse mid-plane of the disc, and just lateral to the lateral margin of the superior articular process.

### Placement of the Intradiscal Needle (240)

The intradiscal needle is specifically designed to facilitate annular puncture and intradiscal access for delivery of the fibrin sealant. The intradiscal needle is manufactured with a slight bend in the distal end to enhance directional control of the needle as it is inserted through the back muscles and into the disc. However a straight intradiscal needle could also be utilized by a practitioner skilled in the art.

The intended path of the intradiscal needle is anesthetized from the subcutaneous tissue down to the superior articular process. The intradiscal needle initially may be inserted under fluoroscopic visualization down to the depth of the superior articular process. The intradiscal needle will be then slowly advanced through the intervertebral foramen while taking care not to impale the ventral ramus. If the patient complains of radicular pain or paraesthesia, advancement of the needle is stopped immediately and the needle is withdrawn approximately 1 cm. The path of the needle should be redirected and the needle slowly advanced toward the target disc. Contact with the annulus fibrosus will be noted as a firm resistance to continued insertion of the intradiscal needle. The needle will be then advanced through the annulus to the center of the disc. Placement of the needle is confirmed with both AP and lateral images. The needle tip should lie in the center of the disc in both views.

Once the needle position is confirmed, a small volume of non-ionic contrast medium may. be injected into the disc. A minimal volume of contrast may be injected to insure avascular flow of the contrast media. If vascular flow is seen, the intradiscal needle should be repositioned and the contrast injection repeated.

### Fibrin Injection (250)

### (a) Loading the Delivery System

After correct placement of the intradiscal needle is confirmed, the reconstituted fibrinogen and thrombin solutions are transferred into the appropriate chambers of the delivery device 100.

### (b) Attaching the Inner Needle Assembly and Intradiscal Needle

The inner needle assembly next is attached to the delivery device 100, and air is expelled from the device. The inner needle assembly with the inner coaxial needle, is next inserted into the intradiscal needle which is already in the center of the target disc, creating a coaxial delivery needle.

### (c) Delivery of the Fibrin Sealant

Placement of the intradiscal needle tip in the center of the target disc is reconfirmed with AP and lateral images. The trigger is then depressed to begin application of fibrin to the disc. Pressure should be monitored constantly when squeezing the trigger. To prevent over-pressurization of the disc, pressure should not exceed 100 psi (6.8 atm) for a lumbar disc.

Each full compression of the trigger will deliver approximately 1 ml of the fibrin to the disc. When the trigger is released, it automatically resets to the fully uncompressed position. Once all of the fibrin has been delivered, the trigger will stop advancing.

Periodic images of the disc should be taken during application of the fibrin to insure that the intradiscal needle has not moved from the center of the disc.

Application of the fibrin to the disc should continue until one of the three following events occurs.

1. The total desired volume of the fibrin is delivered to the disc, usually between 1 - 3 ml, (accounting for any losses within the tubing, needle, system, etc).

2. Continued application of the fibrin would require pressures above 100 psi (6.8 atm).

3. The patient cannot tolerate continuation of the procedure.

After the application of the fibrin is stopped, the intradiscal needle is carefully removed from the patient. Patient observation and vital signs monitoring will be performed for about 20-30 minutes following the procedure.

Extradiscal injection of the fibrin (i.e., injection of fibrin to the exterior of the weakened portion of the herniated disc) may also be carried out using procedures described above. An additional 1-3 ml of fibrin, or the remaining amount available in the delivery device, should be delivered to the external area of the disc that had received surgical decompression. If appropriate, additional amounts of fibrinogen and thrombin may be (prepared and) loaded into the delivery device and delivered to the extradiscal area of the disc annulus. Additionally, fibrin may be injected into other tissues of surrounding spinal structures where benefit from the natural healing milieu may be obtained.

### Example 2

### Re-Hydration of Spinal Disc After Injection of Fibrin

A 66 year old male patient was diagnosed with degenerative disc disease and a herniated L4/L5 disc. At the time of the original diagnosis, discography also revealed IDD in discs L2/L3 and L3/L4, indicating leaking discs with a corresponding loss of disc height. He then received a partial discectomy to decompress the spinal cord and nerve roots on L4/L5 with the Stryker DeKompressor, followed by immediate fibrin injection treatment on date of surgery in the L4/L5 disc and around the exterior surgical site. He received 3 cc of fibrin in the L4/L5 disc nucleus and around the exterior surgical site.

In addition, the patient also received fibrin injections into the L2/L3 and L3/L4 discs to treat the discogenic pain, (IDD). He received 1 cc each, injected into the nucleus of the L2/L3 and L3/L4 discs. (5 cc total for patient). A subsequent discography procedure has revealed a complete sealing of all of the treated discs, along with a return of normal disc height and a complete cessation of pain.

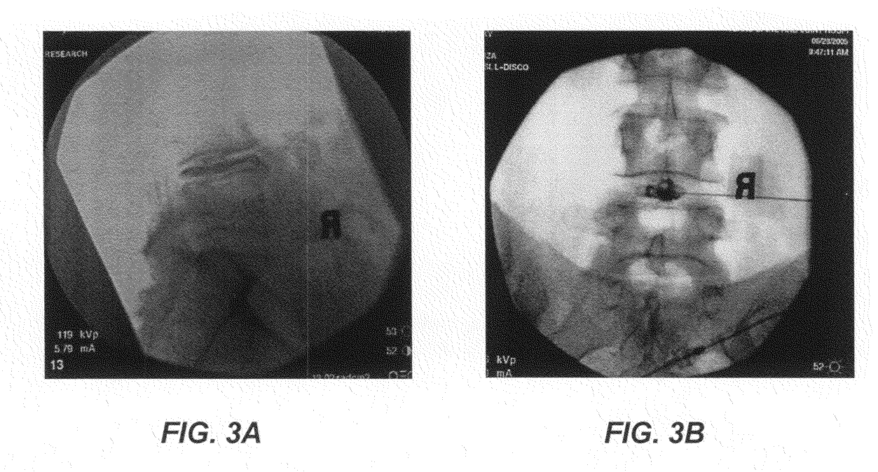

The intradiscal injection of fibrin led to re-hydration of the treated disc. FIG. 3A shows a medial/lateral view of the disc prior to treatment with fibrin sealant, demonstrating annular tears and dehydration. FIG. 3B shows an anterior/posterior view of the same disc at 6 months after the fibrin sealant treatment, demonstrating re-hydration and improved annular structure. The positive results have been maintained for the 2+ years since his procedure, with no further treatment needed.

### Example 3

### Injection into Zygapophysical (Facet) Joints

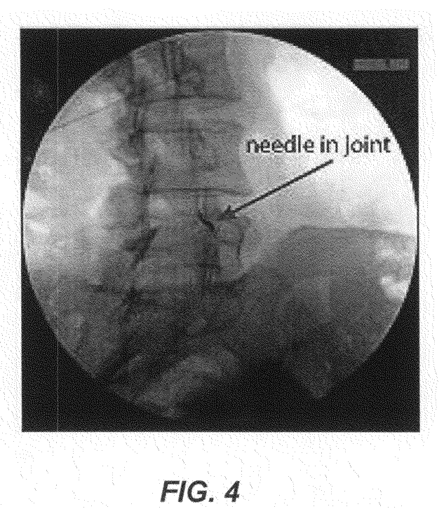

Injection of the zygapophysical joints is performed using the device and procedures described in Example 1. FIG. 4 is a fluoroscopic x-ray of a zygapophysical joint injection. Briefly, following the surgical treatment of the affected areas of the spine, (e.g., discectomy, fusion, ADR, VBR or PVBR), the patient is placed in such a way that the physician can best visualize the facet joints using x-ray guidance. Next, the physician directs the needle, using x-ray guidance into the zygapophysical joint(s). A small amount of contrast (dye) may be injected to insure proper needle position inside the joint space. Then, an effective amount of the biocompatible matrix or biocompatible polymeric compound is injected. One or several joints may be injected depending on location of the patient's usual pain, the degree of surrounding joint degradation and the degree of involvement of the surgically treated spinal area near the zygapophysical joints being treated.

### Example 4

### Stabilization of Discs or Zygapophysical Joints Adjacent to a Surgically Treated Spinal Section with a Dynamic Stabilization or Flexible Spinal System and Injection of Fibrin Sealant

A patient requiring spinal surgery will be prepared for spinal surgery. Upon exposure of the spine, the intended procedure, (e.g., discectomy, fusion, ADR, VBR or PVBR), would be performed, and possibly followed by the installation of the Dynamic Stabilization or Flexible Spinal System. Immediately prior to making final adjustments of the Dynamic Stabilization or Flexible Spinal System, discs, zygapophysical joints and damaged tissues that are immediately adjacent to or relatively near the specifically treated disc, would be injected with fibrin sealant using procedures described in Example 1. Following completion of the injections, any final adjustments would be made to the Dynamic. Stabilization or Flexible Spinal System and the wound would be closed in the normal fashion. Dynamic Stabilization Systems and Flexible Spinal Systems are well known to persons skilled in the art.

### Example 5

### Concurrently Injection of Fibrin into Soft Tissues that are Damaged or at Risk of Being Damaged During a Mini-Open or Open Surgical Procedure

Fibrin is prepared as described in Example 1 and injected into soft tissues that are damaged or at risk of being damaged during a mini-open or open surgical procedure. Examples include small pin-point and button-hole tears within intact neighboring muscles and tendons. Because of adhesive and mechanical limitations, treatment is currently limited to supporting and augmenting the healing of small defects in predominantly intact tissues that maintain primary functional support. These points may also include suture sites and insertion sites at the point of repair for torn muscles (e.g., rotator cuff), torn ligaments (e.g., ACL and collateral knee structures) and tendon repairs (e.g., Achilles tendon). The point(s) of injection are decided by the surgeon performing the surgical procedure. The injection volumes to treat these supporting joint tissues can be determined visually during open surgical procedures range and using spectroscopic information (MRI, sonogram). The volumes of injected biocompatible or polymeric matrix can range from as little as 1 ml to as much as 10 ml or more.

### Example 6

### Injection of Fibrin into Attachment Points and Suture Sites of Soft Tissues

Fibrin is prepared as described in Example 1 and injected into and around the attachment points and suture sites of soft tissues such as meniscal tissue repairs, implants and transplants (e.g., knee), labrum/bucket-handle tear repairs (e.g., glenoid) and reattachment of torn cartilage flaps in almost any articulating joint of the body.

The herein described methods may be used to address various conditions through use of the surgical procedure and biocompatible matrix/biocompatible polymeric compound. The disclosure references particular means, materials and embodiments. Although the claims make reference to particular means, materials and embodiments, it is to be understood that the claims are not limited to these disclosed particulars, but extend instead to all equivalents.

What is claimed is:

1. A method for treating a damaged or degenerated synovial joint or a spinal disc, or a portion thereof, comprising:
injecting into and/or around said damaged or degenerated synovial joint or spinal disc or portion thereof (i) a biocompatible matrix or biocompatible polymeric compound and (ii) a plurality of cells,
wherein at least a portion of said biocompatible matrix or biocompatible polymeric compound is formed in situ at injection site.

2. The method of claim 1, wherein said cells are autologous cells.

3. The method of claim 1, wherein said cells are fibroblasts.

4. The method of claim 3, wherein said fibroblasts are autologous dermal fibroblasts.

5. The method of claim 3, wherein said fibroblasts are autologous oral fibroblasts.

6. The method of claim 3, wherein said fibroblasts are autologous mucosal fibroblasts.

7. The method of claim 3, wherein said fibroblasts are isolated from autologous tendons, ligaments or articular cartilage.

8. The method of claim 1, wherein said cells are chondrocytes.

9. The method of claim 1, wherein said cells are chondrocytes from autologous tendons, ligaments or articular cartilage.

10. The method of claim 1, wherein said cells are allogenic juvenile or embryonic chondrocytes.

11. The method of claim 1, wherein said cells are stem cells.

12. The method of claim 11, wherein said stem cells are autologous mesenchymal stem cells.

13. The method of claim 11, wherein said stem cells are pluripotent stem cells from adult human testis.

14. The method of claim 1, wherein said biocompatible matrix or biocompatible polymeric compound comprises fibrin.

15. The method of claim 1, wherein said synovial joint is selected from the group consisting of zygapophysical joint, costovertebral joint, sacroiliac joint, sacral joint, atlantoaxial joint, hand joints, wrist joints, elbow joints, shoulder joints, temporomandibular (TMJ) joints, sacroiliac joints, hip joints, knee joints, ankle, and foot joints.

16. The method of claim 1, wherein said injecting injects said cells concurrently with said biocompatible matrix or biocompatible polymeric compound.

17. The method of claim 1, wherein said injecting injects said biocompatible matrix or biocompatible polymeric compound prior to injection of said cells.

18. The method of claim 1, wherein said injecting injects said biocompatible matrix or biocompatible polymeric compound after injection of said cells.

19. The method of claim 1, wherein said injecting further comprises injecting into damaged or degenerated soft tissues within or around said synovial joint or spinal disc.

20. The method of claim 19, wherein said soft tissues include muscles, tendons, ligaments, meniscus, labrum and cartilage.

21. The method of claim 1, wherein said injecting injects under fluoroscopic or endoscopic visualization or under direct visualization.

22. The method of claim 1, wherein said portion of the damaged or degenerated portion of the synovial joint being treated is the meniscus and wherein said injecting injects said biocompatible matrix or biocompatible polymeric compound into a meniscus attachment point or suture repair site.

23. The method of claim 1, wherein said portion of the damaged or degenerated portion of the synovial joint being treated is the labrum and wherein said injecting injects said biocompatible matrix or biocompatible polymeric compound into a labrum attachment point, under a flap, or into a suture repair site.

24. The method of claim 1, wherein said portion of the damaged or degenerated portion of the synovial joint being treated is the cartilage and wherein said injecting injects said biocompatible matrix or biocompatible polymeric compound into a cartilage attachment point, beneath a cartilage flap, or into suture repair site.
